(19) United States
(12) Patent Application Publication
(10) Pub. No.: US 2002/0182751 A1
(43) Pub. Date: Dec. 5, 2002

(54) Sperm cell selection system

(76) Inventors: John C. Herr, Charlottesville, VA (US); Kenneth L. Klotz, Esmont, VA (US); Alan B. Diekman, Little Rock, AR (US)

Correspondence Address: John P. Breen, University of Virginia Patent Foundation, Suite 1-110, 1224 West Main Street, Charlottesville, VA, 22903, US

(21) Appl. No.: 10/146,552

(22) Filed: May 15, 2002

Related U.S. Application Data
(63) Continuation of application No. PCT/US00/31771, filed on Nov. 17, 2000.
(60) Provisional application No. 60/166,073, filed on Nov. 17, 1999.

(51) Int. Cl. G01N 33/553

(52) U.S. Cl. 436/526; 210/695; 210/222

(57) ABSTRACT

The present invention relates to a device and method for purifying sperm cells and sperm cell DNA from biological samples containing sperm cells and other cell types. More particularly the present invention is directed to the use of sperm-specific antibodies to purify sperm cells prior to the isolation of the sperm DNA.

### RELATED APPLICATIONS

This application is a continuation of International Patent Application No: PCT/US00/31771, filed on Nov. 17, 2000 and claims priority under 35 U.S.C. §119(e) to U.S. Provisional Patent Application No. 60/166,073, filed Nov. 17, 1999, the disclosures of which are incorporated herein.

### US GOVERNMENT RIGHTS

This invention was made with United States Government support under National Institutes of Health Grant Nos. T32 HD07382, T32 DK07642 and U54 HD29099 and Federal Bureau of Investigations No. 115744. The United States Government has certain rights in the invention.

### BACKGROUND OF THE INVENTION

Sexual assault evidence recovered from a victim is an admixture of various cell types and fluids from both victim and assailant. In cases of vaginal assault, cells originating from the victim include cervical and vaginal epithelial cells, erythrocytes (red blood cells), white blood cells, various vaginal flora, including species of Lactobacillus, Candida, E. coli, as well as cervical mucus and minor contributions from uterine “milk”. Semen, the male component, contains roughly 85% seminal fluid originating from prostate and seminal vesicles, epithelial cells from these organs, spermatozoa, and epididymal fluid (15% of the ejaculate volume) and may contain white blood cells and various bacterial, viral or fungal commensals. In various cases of oral assault, buccal epithelial cells and buccal flora are often present as part of the female component. In cases of anal assault, a variety of intestinal and colonic epithelial cells, secretions, foodstuffs, and bacteria may be present in the female component.

In the mid 1980's, methods of DNA fingerprinting were developed to distinguish male DNA, using Y chromosome probes, and to determine individual specific patterns of DNA using RFLP analysis. DNA extracted from evidence was digested with restriction enzymes, probed on Southern blots with probes directed to genetic loci showing a variable number of tandem repeats (VNTRs) and the resultant restriction fragment length polymorphisms (RFLPs) were detected and catalogued. While yielding results with high discrimination, the VNTR/RFLP method is time consuming, technically demanding, requires significant amounts of starting DNA and is currently limited to specialized laboratories.

The application of the VNTR method to sexual assault evidence also was handicapped by the fundamental problem of extracting DNA specifically from sperm, often termed, the “male component.” Peter Gill, Alec Jeffreys and others developed a technique of preferential lysis, sometimes termed differential lysis, which has gained wide acceptance (Giusti et al., (1986) Journal of Forensic Sciences 3: 409417). This procedure makes use of the observation that the sperm nucleus, which is highly condensed by DNA binding proteins (the transition proteins and protamines) contains a higher proportion of disulfide linkages than do many other cell types, including cervical and vaginal epithelium. This disulfide bonding is undoubtedly an evolutionary adaptation that protects the sperm and the human genome from chemical and physical injuries on the journey to the site of fertilization. For forensic purposes, this disulfide-bonding renders the sperm more resistant to mild solutions containing SDS and the protease, Proteinase K. Treatment of sexual assault evidence with SDS/Proteinase K preferentially lyses vaginal cells while many sperm heads (and their cargo of DNA) remain intact for subsequent extraction in solutions containing SDS/Proteinase K and a reducing agent, such as dithiothreitol (DTT).

The preferential lysis method has proved quite useful when used in conjunction with VNTR/RFLPs because these methods can be used with samples that are not entirely pure. Contaminating DNAs present in an admixture at lower than 10% are usually undetected (Mckeown, Cellmark, personal communication). With its origin in the pre-PCR era, however, the preferential lysis method in many instances may prove unsatisfactory for the current PCR based DNA analyses.

PCR based analysis of DNA can identify the source individual with a high degree of certainty. Many criminals have been identified after comparison of their DNA to the patterns recorded in the convicted offenders database (CODIS). Because of the great sensitivity of the PCR method it is possible to obtain useful data from a small number of recovered sperm, even as few a single sperm. However, with the advent of PCR based reactions and their increased sensitivity, the problem of defining the cellular source of amplified DNA and assigning, beyond a reasonable doubt, that source to the assailant, has proved a more difficult undertaking. The threshold of detection of a contaminating DNA using PCR methods is at least an order of magnitude lower than with VNTR/RFLPs. A need for purer input DNA is thus inherent in the PCR protocol where all DNAs, including contaminating species, undergo amplification prior to analysis.

In short, the power of the PCR strategy lies in its ability to permit analysis of small amounts of isolated DNA, a great benefit when forensic evidence may be in short supply and allowing automation of many samples. The downside lies in the fact that the DNA undergoing analysis must be relatively free of contaminating DNAs. Because the preferential lysis method was developed in a pre-PCR world using the relatively less sensitive VNTR method, new strategies are now needed to refine the purity of the DNA isolated from the “male component”.

The advent of an improved separation method for sperm described herein would save expert witnesses from the problems experienced in explaining the differential lysis procedure. In addition, the method in which sperm are purified to homogeneity using magnetic particles prior to DNA extraction and PCR amplification, would give better information more often, more quickly, and would provide a method that could be easily explained and readily comprehended by jurors, simplifying the interpretation of evidence.

### SUMMARY OF THE INVENTION

The present invention is directed to a method of purifying sperm DNA from a biological sample that comprises multiple cell types. The method comprises selecting male germ cells and separate them from other cell types using the described sperm cell selection system. The DNA can then be recovered from the isolated male germ cells and amplified by a PCR reaction using techniques known to those skilled in the art. In accordance with one embodiment, a sperm immunoselection method is used to isolate highly pure sperm DNA for subsequent amplification. It is also contemplated that the method for isolating the sperm cells will be compatible with an automated robotic device that interfaces with the current PCR probes for short tandem repeats (STR's). The method and device of the present invention provides improvements in the speed and accuracy of handling sexual assault evidence, thus enhancing the development of the National Convicted Offender Database (CODIS).

### BRIEF SUMMARY OF THE DRAWINGS

FIG. 1 is a schematic representation of the steps used in one embodiment to isolate sperm f through the use of paramagnetic particles linked to anti-sperm antibodies.

FIG. 2 is a schematic representation of the steps use in one embodiment to isolate sperm from multiple forensic samples through the use of paramagnetic particles linked to anti-sperm antibodies.

### DETAILED DESCRIPTION OF THE INVENTION

In describing and claiming the invention, the following terminology will be used in accordance with the definitions set forth below.

As used herein, the term “purified” means that the molecule or compound is substantially free of contaminants normally associated with the molecule or compound in a native or natural environment. In particular, purified sperm cell DNA refers to DNA that does not produce detectable levels, or at least does not produce significant detectable levels, of non-sperm cell DNA upon PCR amplification of the purified sperm cell DNA and subsequent analysis of that amplified DNA. A “significant detectable level” is an amount of contaminate that would be visible in the presented data and would need to be addressed/explained during the analysis of the forensic evidence.

As used herein, the term “linkage” refers to the connection between two groups. The connection can be either covalent or non-covalent, including but not limited to ionic bonds, hydrogen bonding and hydrophobic/hydrophilic interactions.

As used herein, the term “secondary antibody” refers to an antibody that binds to the constant region of another antibody (the primary antibody).

As used herein an anti-sperm antibody is an antibody that is specific for sperm cells and promotes at least one of the following activities: agglutination and/or immobilization of spermatozoa, inhibition of tight binding between human sperm and egg vestments, including the cumulus oophorus, the zona pellucida and the oolemma, or blockage of sperm penetration of cervical mucus.

As used herein a sperm-specific antibody is an antibody that binds to an epitope that is unique to sperm cells.

As used herein the term “solid support” relates to a solvent insoluble substrate that is capable of forming linkages (preferably covalent bonds) with various compounds. The support can be either biological in nature, such as, without limitation, a cell or bacteriophage particle, or synthetic, such as, without limitation, an acrylamide derivative, agarose, cellulose, nylon, silica, or magnetized particles.

As used herein the term “magnetic particles” refers to particles that are responsive to a magnetic field.

The present invention is directed to a method and device for purifying sperm cells and sperm cell nucleic acids from biological samples containing a mixture of cell types. In particular, the present invention is directed to the use of sperm-specific antibodies to isolated sperm cells in a separate isolation step before isolating the sperm cell DNA. Isolation of whole sperm cells prior to the isolation of sperm DNA reduces the level of contaminating DNA that occurs using existing procedures.

More particularly, anti-sperm antibodies directed to human sperm surface antigens can be bound to solid supports (such as magnetic particles) to enhance cell separation methods and reduce the presence of contaminating cells in forensic evidence. One preferred sperm-specific antibody is the S19 mAb which recognizes a unique carbohydrate epitope specific to male germ cells. Other sperm-specific antibodies such as MHS 10, which recognizes the sperm acrosomal protein SP-10 (as described in U.S. Pat. No. 5,605,803, the disclosure of which is expressly incorporated herein), and antibodies to SPAN-X, a sperm protein present in nuclear vacuoles and sperm nuclear redundant membranes (as described in PCT/US99/24973, published on May 4, 2000, the disclosure of which is expressly incorporated herein), may also be employed. Other potentially useful sperm surface antigens include C58 or SMARC32. This is not meant to be an exhaustive or exclusive list of potentially useful sperm-specific antibodies and any antibody that is specific for a sperm surface antigen can be used in accordance with the present invention.

The sperm cell selection system of the present invention separates sperm from either dried stains on clothing, from vaginal swabs, from material collected by lavage with physiological saline, or from any suspension which includes sperm cells. In particular the sample containing sperm cells is contacted with a binding substrate comprising an antibody against a sperm specific surface protein and a solid support, wherein said antibody is linked to the solid support. The sample is then incubated with the binding substrate for a time sufficient to allow cells present in the sample to bind to the binding substrate and then the binding substrate is washed with a buffered solution to remove any non-specifically bound material. The remaining bound sperm cells are then lysed and the nucleic acids are recovered using standard techniques.

The solid support may comprise a single solid surface, or more preferably the solid support is in particulate form. In one embodiment, the sperm-specific antibody particles are combined to form a column and the sperm containing sample is run through the column followed by repeat wash solutions.

In another embodiment, the solid support is a magnetic particle selected from one of the several different types of magnetic beads and particles that are currently commercially available and the sperm-specific antibodies are linked to the magnetic particle. In one preferred embodiment the antibodies linked to the magnetic particle comprise one or more monoclonal antibodies selected from the group consisting of MHS 10, S-19, 3C12 and 6C12. In one preferred embodiment the binding substrate comprises the monoclonal antibody S19, covalently linked to a magnetic particle. In accordance with one embodiment the solid support has a plurality of different antibodies linked to the solid support, wherein each antibody binds to a different sperm specific surface protein.

The sperm-specific antibodies of the present invention can be directly linked to functional groups at the surface of the solid support or the antibodies can be attached to the solid support via a linker moiety. In accordance with one embodiment the linker is a secondary antibody that binds to the constant region of the sperm-specific antibody. Alternatively, the linker can be an enzymatically cleavable or photolytic linker. Linkers suitable for use in accordance with the present invention are well known to those skilled in the art.

Advantageously, when the solid support comprises a magnetic particle, the step of separating the sperm cells from the contaminates in the sample, and from the various wash solution can be effected by the application of a magnetic field. For example, in one embodiment after the sperm cells have bonded to the antibody bearing magnetic particles, a source of magnetism can be applied to a exterior surface of the vessel containing the forensic sample. The magnetic force immobilizes the magnetic particles on the interior surface of the vessel thus allowing the remaining contents to be removed (by aspiration for example). The magnetic force can be continuously applied during the washing steps and while the cells are being lysed. More preferably however, after the last wash has been removed from the sample vessel, the magnetic force is deactivated and the particles with the attached cells are resuspended in buffer, and then the cells are lysed.

In accordance with the present invention the disclosed method of isolating sperm cells from a suspension can be automated and a machine can be built using existing technologies that would automatically carry out the necessary steps. In particular, a machine in accordance with this invention would add reagents and remove reagents after immobilizing the sperm and paramagnetic particles on the side of the tube with a fixed magnet or an electromagnet. Furthermore, the machine could also resuspend the cells for the necessary washes, and ultimately isolate them again to the side of the tube with a magnet. The purified sperm would then be delivered in a form suitable for PCR analysis of the DNA. In one preferred embodiment, the purified sperm nucleic acids are PCR amplified using short tandem repeat loci (STR) that have been previously described as providing a high stringency method for identifying individuals.

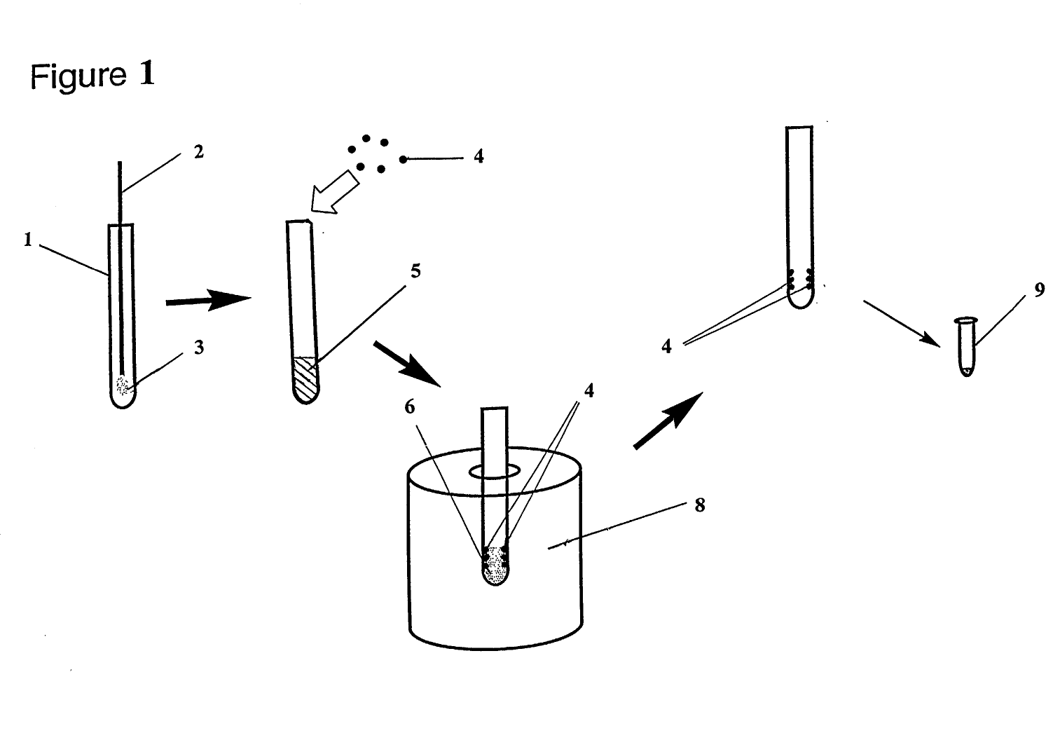

The generalized procedure for isolating sperm DNA from a forensic sample is shown schematically in FIG. 1. Evidence samples collected on cotton swabs (2) are typically stored dried and frozen. In accordance with one embodiment, sperm and other cell types are released from the swab fibers (3) of the cotton swab (2) into suspension in a sample vessel (1) after soaking for about 30 minutes in PBS, teasing of the swab fibers (3) and rinsing with a total of 500 μl PBS. In one embodiment, approximately 25 μl of Ferrofluid paramagnetic particles (4) coated with anti-sperm antibodies are added to the suspension of cells (5) and allowed to incubate for about 10 to about 15 minutes at room temperature. The sample vessel containing the mixture of cells and paramagnetic particles is then inserted into a “magnetic cell separator” (8) for approximately 10 minutes. The magnetic cell separator is a source of magnetism and in one embodiment the separator is cylindrically shape with a bore formed in the cylinder for receiving the sample vessel (1). The magnetic particles (4) and the attached sperm cells are immobilized onto the sides of the sample vessel (1) thus allowing the particles to be washed (for example, washed three times with PBS buffer) and the remaining suspension (6), that includes non-male germ cells, debris and wash buffers is removed from the vessel by aspiration. After the washes the sample vessel (1) is removed from the magnetic cell separator (8) and the paramagnetic particles (4) and sperm are resuspended and transferred to a new tube (9) where the cells are lysed and the nucleic acids recovered using standard techniques. PCR reactions can then be conducted on the isolated sperm DNA and the DNA analyzed by automated sequencing. In one embodiment the PCR reactions are conducted using short tandem repeat loci (STR).

In an alternative embodiment, the sperm cells can be physically removed from the initial forensic sample and transferred to a new vessel. In this embodiment, the forensic sample is contacted with magnetic particles that have sperm-specific antibodies linked to the particle and the particle are incubated with the sample for a time sufficient to bind sperm cells to the particles. A magnetized probe is then placed into the vessel containing the forensic sample and held in contact with the sample for a length of time sufficient to allow binding of the magnetic particles (and the corresponding linked sperm cells) to the probe. The probe is then removed from the vessel, optionally washed to remove non-specifically bound material and the probe is inserted into a new vessel that contains a cell lysis solution. After contacting the probe with the lysis solution for a length of time sufficient to lyse the sperm cells, the probe is removed and the nucleic acid sequences are isolated using standard techniques.

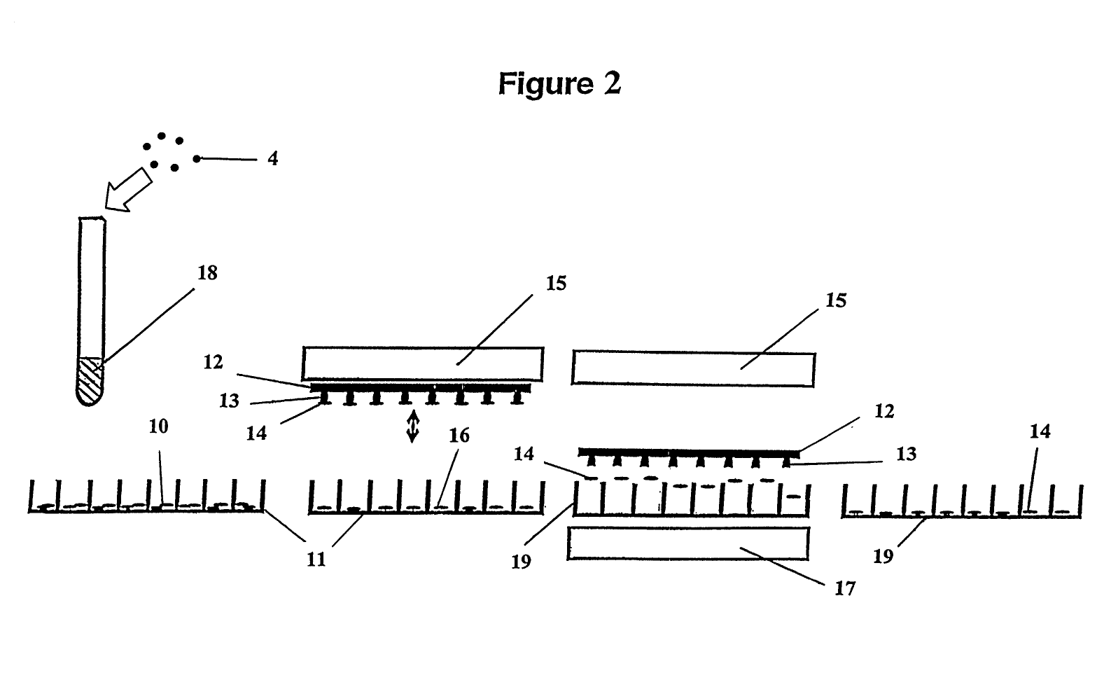

Alternatively, in another embodiment the probe itself is not magnetized, but is composed of material that can have magnetism conferred to it by an adjacent fixed magnet or electromagnet. When the probe is in sufficient proximity to the magnet to have magnetism conferred to it, the probe is referred herein as being “magnetically coupled” to that magnet. In this manner the magnetic beads can be released from the probe after the last wash is completed by either stopping the current flow to the electromagnet or by physically separating the fixed magnet/electromagnet from the probe (see FIG. 2). To assist in the removal of the magnetic particles from the probe after the probe is physically moved to the second vessel, a second source of magnetism can be applied to the exterior wall of the second vessel after the magnetic source that is conferring magnetism to the probe is removed. The ability to release the magnetic bead from the probe after the washing steps allows for the isolation of viable intact sperm cells.

In accordance with one embodiment intact viable sperm can be recovered from a complex mixture and concentrated through the use of the binding substrates of the present invention. For example, one or more sperm-specific antibodies can be linked to a magnetic particle via an enzymatically cleavable or photolytic linker. Once the sperm cells have bound the sperm-specific antibodies, the binding substrates can be isolated using the techniques described herein, thus purifying and concentrating the sperm cells. The individual sperm cells can then be released from the solid support by disrupting the linkage between the antibody and the solid support (either by enzymatic cleavage or by photo-reaction of the photolytic bond). In one preferred embodiment the sperm specific antibody used is a recombinant monoclonal antibody that is monovalent to prevent crosslinking and agglutination of the sperm cells. An example of such an antibody is the recombinant S19 antibody, RASA, described in International Application No. PCT/US0019843, the disclosure of which is expressly incorporated herein. Such a procedure can be used to isolate viable sperm cells and to concentrate sperm cells present in a sample. Such methods can be used to overcome infertility problems associated with low sperm counts.

The methods of the present invention can also be used to isolate a single sperm cell from a suspension of cells released from a swab collected as evidence and containing a complex mixture of sperm cells. This would allow for the isolation of a single sperm cell's DNA and the identification of an individual that contributed to the mixed sample. To isolate a single sperm cell, a device is provided that utilizes the specificity of an anti-sperm antibody coupled to paramagnetic particles to isolate single sperm cells. More particularly, the device used in this procedure comprises a video camera for tracking individual sperm cells in a forensic sample and an electromagnetic “sperm picker”. In one embodiment the sperm cells are fluorescently labeled to assist in identifying individual sperm cells. The video camera is in electronic communication with a computerized control unit that allows one to position an electromagnetic probe, having suitable dimensions, to contact a single sperm cell and reversibly bind the magnetic particle to the probe.

The isolation of single sperm cells (and subsequent isolation and amplification of that sperm cell's DNA) would be particularly useful in cases where sperm from more than one male were present. In this design sperm with adherent paramagnetic particles would be “picked” from a suspension with a magnetic probe only a few microns in diameter. The magnetic field would be generated by an electromagnet that could be turned on to pick up a sperm and turned off to release it into another tube for PCR analysis. A fixed magnet surrounding the PCR tube would pull the sperm into the PCR tube when it was released from the probe. Individual sperm for analysis could be identified by an individual operator with the aid of a microscope or an automated system could be developed that would use videomicroscopic images linked to a computer to control the Sperm Picker. Sperm would either be identified by shape and size parameters or they could be fluorescently tagged in the sample using an anti-sperm antibody conjugated to a fluorescent dye to facilitate identification.

To isolate a single sperm cell from a complex mixture, the sample is first contacted with binding substrate for a time sufficient to allow cells present in the sample to bind to the binding substrate. Preferably the binding substrate comprises an antibody against a sperm specific surface protein and a magnetic particle, wherein said antibody is covalently linked to the magnetic particle. The binding substrate is then washed with a wash solution to remove any non-specifically bound material and the isolated sperm cells are placed in a droplet on a slide. The electromagnetic probe (sperm picker) is then positioned to capture the target sperm cell. The sample may have to be subjected to serial dilutions to produce a sample that has the optimal concentration of sperm cells to allow contact of the probe with a single sperm cell. The electromagnetic probe binds to the magnetic particle and the probe is then transferred to a reaction vessel where the electromagnet is deactivated to release the magnetic particle and its linked sperm cell into the vessel. To assist the release of the magnetic particle into the reaction vessel, the exterior surface of the reaction vessel can be contacted with a source of magnetism (a fixed magnet or another electromagnet) after the probe electromagnet has be deactivated. The electromagnetic probe is then removed and the sperm cell is lysed and subjected to standard PCR amplification of the sperm cell DNA to allow sufficient material for forensic analysis, including sequencing of the DNA.

Advantageously the use of magnetic particles allows the system of isolating sperm specific DNA to be automated. In particular, robotic arms can be used to add, remove or transfer fluids from one vessel container to another. The computer software and the mechanical hardware necessary for conducting such automation is known to those skilled in the art and has been previously described, for example see U.S. Pat. Nos. 5,366,896 and 5,128,103 the disclosures of which are expressly incorporated herein.

In accordance with one embodiment of the present invention a device for isolating sperm cell DNA from a sample comprising sperm cells and other cell types comprises a robotic arm coupled to an electromagnet, wherein the robotic arm is programed to place the electromagnet into a first compartment that contains the forensic sample, and then remove and place the electromagnet into a second compartment where the cells are lysed. In one embodiment the device further comprises a metallic pin magnetically coupled to said electromagnet. The device can be further provided with a second magnetic source (either a fixed magnet or an electromagnet) located outside the second compartment but in close enough proximity to the second compartment so as to impart a magnetic force on the contents of the second compartment. This magnet is used to assist in removing the magnetic particle from the first electromagnet after that electromagnet has been deactivated.

The device can be further provided with automated means for dispensing liquid into and withdrawing liquid from the first and second compartments. These automated dispensing and withdrawing means may comprise a system of positive and negative pressure pumps that direct fluids through tubes to the first and second compartments. Alternatively, the automated dispensing and withdrawing means may comprise one or more dispensing tubes attached to separate robotic arms wherein the dispensing and withdrawal of fluids to specific compartment is programmed.

In accordance with one embodiment a sperm immunomagnetic selection device is provided for high throughput automatic processing as shown in FIG. 2. The device uses a multichambered microtitre plate (11), for example a 96 well microtitre plate, wherein each well contains a forensic sample (18). Approximately a 100-200 μl suspension of sperm and other cell types extracted from evidence swabs, or other sources of sperm containing material, are placed in the wells of the microtitre plate (11). A saturating amount of sperm-specific antibody (for example the S19 mAb) is then added and allowed to incubate with the sample for about 10 to about 30 minutes. A secondary antibody specific for the sperm-specific antibody is then added, wherein the secondary antibody is covalently bound to the magnetic particle (4), and incubated for a sufficient amount of time to bind to the sperm. When the S19 antibody (generated in mice) is used as the primary sperm-specific antibody, the secondary antibody can be a goat anti-mouse IgG coated Ferrofluid particle that is commercially available. Alternatively the sperm-specific antibody can be directly bound to the magnetic particle or bound through some other linker selected from those known to the skilled practitioner. The magnetic particles (4) are incubated with the forensic sample (18) in a suspension (10) for a sufficient length of time to bind sperm cells present in forensic sample (18) and produce magnetic particle coated sperm (14).

To separate sperm cells from non-sperm cell types (16) and sample contaminates, the forensic samples (18) are contacted with a plurality of extensions (13) that extend perpendicularly from the bottom of a magnetized plate (12). The extension (13) are configured to simultaneously fit within the wells of a microtitre plate (11) and contact the solution contained within the wells. In accordance with one embodiment the plate has 96 metal pins that have magnetism conferred by an adjacent magnetic source (15), for example a fixed magnet or an electromagnet. The extensions (13) are inserted into the wells while the magnetized plate (12) and extensions (13) are magnetically coupled to the magnetic source (15) so that the magnetic particles (4) bind to the extensions (13). The magnetized plate (12) bearing the magnetic particle coated sperm (14) is then removed from the wells of the microtitre plate (11), optionally washed to remove non-specifically bound material, and transferred to a new microtitre plate (19). The cells can then be lysed to release their nucleic acids and the nucleic acids recovered using standard techniques.

Alternatively, the magnetic particles (4) can be released from the extensions (13) by removing the magnetic source (15) to the magnetized plate (12) and extensions (13) and placing second magnet (17) under the new microtitre plate (19). Thus the magnetic particle coated sperm (14) are released from the extensions (13) and attracted to the bottom/sides of the wells in the new microtitre plate (19). The extensions (13) are then removed from the wells, the isolated sperm cells lysed and the nucleic acids recovered using standard techniques. The recovered nucleic acid sequences are then amplified using the polymerase chain reaction (PCR) and the DNA analyzed by electrophoresis or automated sequencing.

In one aspect of the present invention a device for isolating sperm cell DNA from a sample comprising sperm cells and other cell types is provided. The device comprises an electromagnet magnetically coupled to a plate having a plurality of vertically extending pins. The electromagnet itself is attached to a robotic arm wherein the robotic arm is controlled by a computer that provides automated means for moving the electromagnet from a first compartment to a second compartment, wherein the first compartment is formed for receiving a forensic sample and the second compartment is formed for receiving a cell lysis solution. The device is further provided with automated means for dispensing liquid into and withdrawing liquid from the first and second compartment. In a modified version of this embodiment the device is provided with a third compartment that is formed for receiving a buffered wash solution. In this device the robotic arm is programmed to move the electromagnet from the first compartment (that contains a sperm sample) to the compartment containing the buffered wash solution and then to the compartment containing the cell lysis solution.

In accordance with one embodiment, the monoclonal antibody is directed against a unique human sperm surface carbohydrate epitope located on sperm agglutination antigen-1 (SAGA- 1). For example one suitable antibody is S19 or recombinant mini-antibody derivative of S19 (RASA). SAGA-1 is synthesized in the principal cells of the epididymis, is specific to the male reproductive tract of humans and higher primates, and is inserted by way of a GPI anchor into all domains of the human sperm surface: head, midpiece and tail. Thus this antigen offers an excellent target for developing methods for separating sperm cells from other biological materials. In order to maximize the binding capacity of the paramagnetic particles for sperm, it is necessary to use a purified preparation of S19 monoclonal antibody that is free of other immunoglobulins and/or contaminating proteins. A suitable purification strategy is described in Example 1.

### EXAMPLE 1

Purification of the S19 Monoclonal Antibody

In order to maximize the binding capacity of the magnetic particles for sperm, it is necessary to use a purified preparation of S19 monoclonal antibody that is free of other immunoglobulins and/or contaminating proteins. Purified S19 mAb was produced at the University of Virginia. Based on preliminary trials, the production of 1 gram of S19 mAb requires a total of 150 iMABTM Gas Permeable Bags (Diagnostic Chemicals Ltd, Charlottetown, PE, Canada) filled with 500 ml CCM-1 serum-free medium (Hyclone), each inoculated with 1×10⁸ cells from the MHS8 clone. The hybridoma cells are cultured at 37° C. in the presence of 5% CO₂ for approximately 3 weeks until the cell density has increased to its maximum and then declined to 10% or fewer viable cells. The culture medium is harvested and the resulting 75 liters with an expected concentration of 13 mg/L IgG is concentrated 15 fold with 10,000 Dalton molecular weight cut off Centricon filters (Amicon) to a total of 5 liters with an expected concentration of 195 mg IgG per liter. The antibody is purified using a recombinant Gamma Bind Plus Protein G affinity column (Pharmacia) on a Shimatsu HPLC system.

It has been demonstrated that a higher yield of 16 mg IgG/500 ml culture is possible if the hybridoma is grown in Iscove's medium with 10% fetal bovine serum instead of CCM-1 serum-free medium. The purity of the resultant antibody is confirmed by SDS-PAGE electrophoresis. The acrylamide gel is stained with Coomassie Blue to visualize any contaminating proteins that might be present. The activity of the antibody is determined by the limiting dilution in an ELISA assay (Engvall et al. 1972) where the ELISA plate is coated with human sperm or sperm extract. The reactivity with native SAGA-1 on sperm is also confirmed by indirect immunofluorescent staining of sperm following the method of Diekman et al (1997) Biology of Reproduction 57: 1136-1145.

### EXAMPLE 2

S19 Monoclonal Antibody Conjugated to Paramagnetic Beads Can Be Used to Capture and Isolate Human Sperm.

It has been demonstrated that S19 recognizes a unique carbohydrate epitope that is present on the entire surface of the human sperm and that S19 will freely bind to sperm in suspension causing their agglutination (see U.S. Pat. No. 5,830,472, the disclosure of which is expressly incorporated herein). The S19 antibody has been conjugated to paramagnetic beads (Dynal, Inc., Lake Success N.Y.) and these beads have been used to separate human spermatozoa from a suspension. Dynal supplies two forms of the 4.5 micron diameter paramagnetic beads.

Dynabeads M-450 are tosylactivated beads that allow the direct conjugation of S19 mAb to the bead but without regard to orientation of the antibody on the bead. Dynabeads-450 covalently bind the S19 antibody via p-toluene-sulfonyl chloride (tosyl) reactive groups which will react with any protein containing primary amino or sulfhydryl groups. Dynabeads-450 are best suited for the production of sperm binding beads using RASA, the recombinant anti-SAGA-1 antibody lacking an Fc region (RASA is fully described in International Application No. PCT/US00/19843, the disclosure of which is expressly incorporated herein). Dynabeads Pan Mouse IgG bind the S19 mAb via a secondary human anti-mouse IgG antibody that is specific for the Fc region of the IgG molecule. Binding of the bead to the Fc region leaves both reactive sites of the IgG molecule free to bind to spermatozoa. The increased bivalent binding results in more sperm bound to the bead and those sperm bound more tightly.

Tosylactivated Beads Bind Monoclonal Antibody S19.

Dynabeads-450 tosylactivated are uniform, superparamagnetic, polystyrene beads coated with a polyurethane layer. The polyurethane surface is activated by p-toluene-sulfonyl chloride to provide reactive groups for covalent binding of proteins such as antibodies containing primary amino or sulphhydryl groups. They are 4.5 um in diameter and are supplied at a concentration of 4×10⁸ beads/ml. The S19 IgG used for coating the beads was purified on a protein G column from the supernatant of a MHS8 clone grown in CCM-1 serum-free medium. Following the manufacturer's recommendations, 100 ul of Dynabeads-450 Tosylactivated (4×10⁷ beads) were incubated with 20 ug of purified IgG S19 antibody (8 ul of 260 ul/ml stock) in 0.1 M sodium phosphate pH 7.4 for 10 minutes at 37° C. with occasional mixing. They were then incubated 16 hours at 4° C. after adding 20 ul of 0.5% BSA and 2 ul of 1% sodium azide. The beads were washed in PBS plus 0.1% BSA, blocked 4 hours at 37° C. in 0.2 M Tris plus 0.1% BSA, washed again in PBS plus 0.1% BSA and stored at 4° C. in the same with 0.02% sodium azide. Negative control beads were prepared in the same manner except that they were incubated with 0.1 M phosphate buffer pH 7.4 instead of with S19 mAb. In order to confirm the coating of the S19 mAb on the beads, they were reacted with TRITC labeled F(ab )2 donkey anti-mouse IgG (Fc region specific) at 1:50 dilution for one hour at room temperature, washed in PBS and viewed with a Zeiss Axioplan fluorescent microscope. The fluorescence of the beads was observed, indicating that the monoclonal antibody S19 coated the magnetic bead.

Preparation of S19 Coated Dynabeads Pan Mouse IgG.

Dynabeads Pan Mouse IgG are mono-sized, superparamagnetic, polystyrene beads coated with a monoclonal antibody specific for the Fc region of all mouse IgGs. The human IgG monoclonal is attached covalently to Dynabeads-450. The Pan Mouse IgG antibody does not cross react with human, rat, rabbit, guinea pig, sheep, goat, or hamster IgG. Dynabeads Pan Mouse are supplied as a suspension containing 4×10⁸ beads/ml in PBS with 0.1% BSA and 0.02% sodium azide.

To prepare S19 coated beads, a mixture of 100 ul beads and 4 ug purified S19 IgG was incubated for 90 minutes at 37° C. with gentle agitation. Negative control beads were prepared by incubating 4 ug of a saturated ammonium sulfate precipitated enriched IgG fraction from an IgG null ascites. The coated beads were washed, restored to 100 ul volume, and stored in PBS plus 0.1% BSA and 0.02% sodium azide at 4° C. until use.

Sperm Released from a Fresh or Dried Swab are Captured by S19 Coated Dynabeads Pan Mouse.

An ejaculate was diluted with Ham's F10 medium to a low final sperm concentration of 5×10⁵/ml. A sample simulating a forensic sample was collected with a swab made of synthetic material which absorbed 130 ul fluid. Sperm were washed from the swab with 260 ul of PBS with 0.1% BSA and 0.05% Tween 20. Five ul of S19 coated beads were added to the sperm suspension and allowed to incubate for 60 minutes at 37° C. with gentle agitation. The magnetic beads were separated from the fluid and gently washed in PBS/BSA/Tween 20 using a Magna-Sep (Gibco-BRL) device. The Magna-Sep uses magnets to attract the beads to the side of a microfuge tube permitting convenient retrieval of cells bound to the beads. The beads were examined using phase contrast with a Zeiss Axiophot microscope.

The magnetic beads bound sperm in all regions of the sperm cell membrane from head to tail. No other cell types were present. Large agglutinated masses of sperm and beads were also observed. These results indicate that development of a S19 mAb bound sperm separation method is feasible.

### EXAMPLE 3

Optimization of Sperm Isolation Procedure

The success with the Dynabeads proves the principle of separating sperm with an S 19-coated paramagnetic bead. To optimize the method further, a different size and shape of magnetic particle has been utilized. Immunicon Ferrofluids (Immunicon Corp, Huntingdon Valley, Pa.) consist of magnetic cores (magnetite crystals Fe₃O₄) coated with a covalently linked polymeric material. Although they have many other uses (immunoassay, molecular biology, viral capture, etc.), these reagents have been primarily designed and optimized for cell separations directly from whole blood. Immunicon provides two types of ferrofluids, amino/carboxy polymer coated conjugated ferrofluids and common capture ferrofluids. The amino/carboxy polymer coated ferrofluids are used for direct coupling of a ligand such as S19 mAb. The common capture ferrofluids employ a secondary antibody and can be used for separation with any appropriate monoclonal antibody. The magnetic reagents exhibit low levels of non-specific binding and the magnetic separation procedure has little carryover of contaminating cells (<0.01% in whole blood). Separations can be carried out in standard 12×75 mm test tubes.

Immunicon magnetic particles are irregular in shape and 120-150 nm in size. These submicron particles offer several advantages. Large magnetic particles that sink at a faster rate than small particles require constant agitation to ensure contact with targeted material. Too strong an agitation results in shearing of the cells from the particles. Immunicon's Ferrofluids contain small particles diffused throughout the sample solution, increasing target capture rate while decreasing incubation time. Smaller magnetic particles move more slowly toward the magnet so there is less shear force to dislodge the captured sperm compared to either of the much larger 4.5 um Dynabeads. The irregular shape adds more surface area, increasing their binding capacity compared to larger spherical particles.

There are several potentially useful strategies for coupling a mAb to a paramagnetic particle. Linking the S19 mAb to a paramagnetic particle that has been coated with a secondary antibody to the Fc region of mouse IgG offers the advantage of optimally presenting the bivalent immunoreactive sites of the IgG molecule to the sperm surface, but use of the secondary antibody may result in agglutination of the particles. Paramagnetic particles coated with avidin to bind biotinylated antibody may also be used. The best way to avoid agglutination of particles and to avoid nonspecific reactions of the particles to unwanted cell types may be to avoid the use of secondary coupling reagents and instead utilize paramagnetic particles that allow direct conjugation of the antibody similar to those supplied by Immunicon. These magnetic particles possess a proprietary activated silica coat that allows direct coupling via amine groups of the antibody to the bead.

Coating Particles with S19 mAb:

Immunicon supplies Ferrofluid coated with a goat antibody specific for the Fc region of mouse IgGs. The beads are supplied as a colloidal suspension with a density of 4×10¹¹ particles per mg iron. The binding capacity is approximately 50 ug mouse IgG per mg Ferrofluid. To prepare particles coated with S19 mAb, 50 ug of purified S19 was mixed with 100 ul of Ferrofluid and incubated for 90 minutes at 37° C. with gentle agitation. The coated beads were washed using the Immunicon magnetic separator and stored in PBS plus 0.1% BSA and 0.02% sodium azide at 4° C. until use. The small irregularly shaped paramagnetic particles bound tightly to sperm and the separation could occur under normal laboratory conditions using the magnetic cell separator and by pipetting and washing the particle bound sperm.

Direct coupled S19-Ferrofluid may be prepared with amino polymer coated particles by Immunicon using their proprietary process. Alternatively, standard covalent coupling chemistry to coat the amino polymer particles with S19 mAb may be employed.

To test the ability of sperm-specific antibody bearing magnetic particles to bind to sperm cells, the antibody was fluorescently labeled and combined with a sample containing sperm cells. A kit from Molecular Probes, Inc. was used to conjugate the fluorescenttag Alexa 568 to the S19 mAb. S19 was purified on a protein G column from the supernatant of a MHS8 clone grown in Iscove's medium with 10% fetal bovine serum. Ferrofluid particles coated with goat anti-mouse IgG, Fc region specific, from Immunicon, Inc. were coated with S19 mAb that had been fluorescently labeled with Alexa 568. Washed sperm at a concentration of 1×10⁶/ml in 100 ul PBS, 0.1% /BSA was incubated with 10 ul fluorescent S19 coated Ferrofluid particles for 30 minutes at room temperature. The magnetic particles and bound sperm were washed twice in PBS with the Immunicon Magnetic Separator and viewed with a Zeiss Axioplan microscope. Differential interference microscopy was used to show the location of paramagnetic particles at the sperm surface, while immunofluorescence microscopy showed fluorescence of the paramagnetic particles.

Separation of Sperm Using Paramagnetic Particles Coated with S19 via a Secondary Antibody:

In preliminary studies sperm suspensions from fresh semen samples or dried swabs were used as the source material. When fresh semen was used, a 1 ml sample of semen diluted 1:100 in 0.01 M phosphate buffered saline (PBS) pH 7.2 was mixed with 20 ul of goat anti-mouse IgG Fc specific Ferrofluid coated with S19 mAb in a 12×75 mm tube and allowed to incubate at room temperature for 30 minutes with gentle agitation. Forensic samples typically consist of vaginal, oral, or anal cotton swabs that are dried and stored frozen until assayed. Sperm were extracted from a dried cotton swab containing semen diluted 1:100 in PBS by soaking the swab in 250 ul PBS for 30 minutes, teasing the outer fibers of the swab with fine forceps and rinsing with an additional 250 ul PBS. The suspension of released sperm was incubated with 20 ul goat anti-mouse IgG Fc specific Ferrofluid coated with S19 mAb in a 12×75 mm tube and allowed to incubate at room temperature for 30 minutes with gentle agitation.

After incubation, the tube containing the suspension was inserted into a 12 mm Immunicon magnetic separator where the ferrofluid was allowed to separate for 10 minutes. After separation was complete, the supernatant was aspirated taking care not to disrupt the material collected on the tube wall. The magnetic particles and attached sperm were washed once with PBS again taking care not to disrupt the sperm-bead complexes on the tube wall. The tube was then removed from the separator and the collected material evaluated with a Zeiss microscope to determine the presence of sperm suitable for processing for PCR analysis of DNA.

Human sperm cells coated with S19 mAb were successfully isolated from a suspension with Ferrofluid paramagnetic particles coated with the secondary antibody goat anti-mouse IgG (Fc region specific). Two million washed sperm were incubated with a saturating amount of 50 ug S19 mAb in 0.5 ml PBS, 0.1% Tween 20, 3.0% normal goat serum (NGS). The S19 was a saturated ammonium sulfate precipitate of ascites fluid. After incubation for 15 minutes at room temperature the sperm were washed by centrifugation with PBS, 0.1% Tween 20, 1% NGS and resuspended in 500 ul of the same. 10 ul Ferrofluid particles coated with goat antimouse IgG, Fc specific were added to the sperm suspension and incubated for 15 min at room temperature. Magnetic particles and bound sperm were isolated from the suspension washed with PBS/Tween/BSA using an Immunicon Magnetic Separator. They were resuspended in 100 ul PBS and viewed with a Zeiss Axioplan microscope confirming the ability of the binding substrate to bind sperm cells.

Separation of Sperm Using Paramagnetic Particles Directly Conjugated to S19:

Sperm have been isolated from suspension using paramagnetic particles directly conjugated to S19. Purified S19 IgG was coupled to Ferrofluid paramagnetic particles by Immunicon using their proprietary method. It is also possible to conjugate antibody to Ferrofluid particles using non-proprietary coupling chemistries. Washed sperm that had been stored frozen were diluted to a concentration of 2 million per ml in PBS with 10% normal goat serum. The sperm were incubated with 20 ul Ferrofluid directly coupled to S19 mAb for 15 minutes at room temperature before isolation and washing 3 times with PBS using the magnetic cell separator. A large number of sperm were recovered and examined with a Zeiss Axiophot microscope. Paramagnetic particles were seen bound to the sperm. After storage for 16 hours at 4° C. many sperm were seen to be completely covered with paramagnetic particles over the entire sperm. Paramagnetic particles directly coupled to S19 mAb are effective in separating human sperm cells from a suspension.

Washed human sperm that had been stored frozen were diluted to a concentration of 2 million per ml in PBS with 10% normal goat serum. The sperm were incubated with 20 ul Ferrofluid directly coupled to S19 mAb for 15 minutes at room temperature before isolation and washing 3 times with PBS using the magnetic cell separator. A large number of sperm were recovered using an Immunicon Magnetic Separator and examined with a Zeiss Axiophot microscope. Paramagnetic particles were seen bound to the sperm. After storage for 16 hours at 4° C. many sperm were seen to be completely covered with paramagnetic particles over the entire sperm.

As an alternative to the use of Immunicon Ferrofluids to separate sperm from epithelial cells and body fluids, the sperm suspension can first be incubated with saturating amounts of biotinylated S19 mAb. After 15 minutes at room temperature the sperm cells will have been coated with the S19 mAb and may be selected from the suspension with avidin coated Ferrofluid particles supplied by Immunicon following the manufacturers standard protocol. In addition to Immunicon Ferrofluids, there are several other magnetic particles available which may prove more useful. Larger spherical Dynabeads have already been shown to be effective in binding sperm and separating them from a suspension. It may be possible to improve the method and produce a stronger bond between the sperm and the bead using cross linking agents after the sperm have been bound. PromoCell (Heidelberg Germany) supplies Quantum Biomagnetics magnetic particles which can be coated with an antibody or other protein. The particles are irregularly shaped providing a greater surface area for binding than spheres. They are available in either 50 nm or 250 nm size either smaller or larger than Ferrofluid particles. It may be that one of these sizes is more optimum for separating sperm cells. Other companies offering paramagnetic beads include Beckman Coulter (1 um diameter) and Roche Molecular Biochemicals (1 um diameter).

### EXAMPLE 4

DNA Analysis with PCR Using Short Tandem Repeat Loci:

PCR using short tandem repeat loci (STR) has proved to be a high stringency method for identifying individuals. These loci are simple tandemly repeated sequences of 1-6 base pairs (bp) in length which vary among individuals in the number of repeats displayed. Repeated sequences can be identical (simple) or complex. STRs appear in the genome every 6-10 kilo bp and are easily identifiablethrough PCR analysis. As PCR is a procedure that utilizes amplification, it requires very little DNA starting material. Fluorescent primers to STRs located on various chromosomes generate discreet bands that are specific to an individual. Fluorescence-based PCR technology followed by automated sequence analysis provide the tools for creating a DNA fingerprint for an individual.

PCR Testing Procedure:

DNA is collected from sperm cells separated by the procedure described above using procedures modified from Glassberg et al. (1985), see Giusti et al., (1986) Journal of Forensic Sciences 3: 409-417. Resulting DNA will be run through a standard PCR procedure to identify individuals using short tandem repeat (STR) loci. Sperm cells will be washed three times in PBS with 2% Sarkosyl followed by centrifugation at 3600 rpm at 4° C. Washed samples are resuspended in PBS containing 100 ug/ml proteinase K and 1% sodium dodecyl sulfate (to remove other cell types, if present), then incubated for 4 hours at 65° C. with mild agitation. Sperm heads are pelleted by centrifugation as above and lysed following resuspension in PBS/2% Sarkosyl/100 ug/ml proteinase K/10 mM dithiothreitol/25 mM EDTA and incubating overnight at 37° C. (or 55° C. for 4 hours). Precipitating out contaminating proteins with 6 M NaCl and centrifuging at 13,000× g for 10 minutes will purify the resulting sperm DNA. The purified DNA is precipitated with absolute ethanol and mixed until genomic DNA becomes visible. A sterile pipette tip is used to collect the DNA that will be washed with 70% ethanol before being dissolved in water at 37° C. for 10 minutes. The concentration of the sample will be calculated by an A_{260/280} reading and diluted (if necessary) to 100 ug/ml.

PCR amplification will be run in 50 ul total volume containing 1-10 ng of genomic DNA, 50 mM KCl, 10 mM Tris-HCl pH 8.3, 1.5 mM MgCl₂, 200 mM of each dNTP, 0.25 mM primers (D21S11, D20S85, D18S51, HUMVWFA31/A, HUMTHO1, D6S502, HUMFIBRA, HUMAMGXA, HUMAMGY as reported in Oldroyd et al. (1995) Electrophoresis 16: 334-337, and 1.25 units AmpliTaq DNA Polymerase (Perkin-Elmer, Norwalk, Conn.). The following PCR program will be employed: 94° C. for 2 min., 30 cycles of 94° C. for 30 sec., 58° C. for 75 sec., 72° C. for 15sec., and a final 10 minute cycle at 72° C. PCR fragments will be combined with formamide and an internal size standard (such as bacteriophage lambda DNA) before being denatured for 2 min. at 90° C. and run on a 6% denaturing acrylaminde gel. Gels will be run on an ABD 373A GeneScanner and their fragment sizes determined automatically.

### EXAMPLE 5

Single Cell PCR.

A further refinement of the above techniques employing higher stringency would be the use of PCR from single sperm cells (Lien, et al, (1993) Genomics 16: 41-44). This would be particularly useful when the number of sperm recovered is very low. It would also be an important technique to use when it was necessary to differentiate between sperm from more than one individual in the evidence sample. In order to more easily locate single sperm cells and facilitate their retrieval, they can be labeled with fluorescent conjugated S19 either before or after capture by goat anti-mouse IgG coated magnetic beads (Immunicon). S19 will be directly conjugated with Alexa 568 fluorescent dye (Molecular Probes) following the manufacturer's protocol.

Sperm cells bound to the magnetic particles will be washed as above, and Alexa 568-conjugated S19 added to the mix. Unbound Alexa 568-S19 will be washed off the magnetic particles with PBS. Alternatively the sperm can be fluorescently labeled before capture with magnetic particles. The evidence sample is incubated with Alexa 568-conjugated S19 at 1:50 dilution for 30 minutes at room temperature. The cells are then centrifuged at 500× g and washed 3 times to remove S19 not bound to sperm. Goat anti-mouse IgG coated beads are then used to capture the sperm coated with fluorescently-labeled S19 mAb. With either method, washed, fluorescently-tagged sperm cells will be visualized on a glass slide using a Zeiss Axioplan microscope equipped for UV fluorescence.

Individual cells will be picked up using pulled glass pipettes in a micromanipulator (Moskaluk and Kern 1997). Alternatively, an electromagnetic sperm cell picker can be used to pick individual sperm cells through the use of the activated magnet. Isolated sperm cells will be lysed with 2.5 ul lysis buffer (200 mM KOH, 50 mM dithiothreitol) for 20 min. at 65° C. and neutralized with 2.5 ul buffer containing 900 mM Tris-HCl pH 8.3, 300 mM KCl, and 200 mM HCl. PCR will be performed as outlined above, and the samples analyzed.

The sperm cell selection system may be utilized in a number of different effective configurations. In accordance with one embodiment, the sperm cell selection system can be used with commercially available magnetic separators to obtain a sample of purified male germ cells using antibodies specific for proteins on the sperm surface. Swabs collected as evidence are soaked in PBS and teased apart to release the adherent cells into suspension. Paramagnetic particles coated with anti-sperm antibodies bind to the target sperm cells. Alternatively paramagnetic particle coated with a secondary antibody bind to sperm which have been incubated with the anti-sperm antibodies. Sperm cells coated with particles are separated from the suspension using a variation of commercially available magnetic separators.

### EXAMPLE 6

High Throughput Automated Analysis of Forensic Samples

In addition, a high throughput automated device for analyzing samples can be modeled on the work published by Hancock and Kemshead (1993). Sperm cells can be isolated simultaneously from 96 well microtitre plates and transferred to new microtitre plates for the PCR reaction (FIG. 2). In one embodiment, 100-200 ul of sperm and cell suspensions extracted from evidence swabs would be placed into wells of the microtitre plate. To this would be added a saturating amount of S19 mAb. After a short time for incubation of 10-30 minutes the goat anti-mouse IgG coated Ferrofluid particles would be added and allowed to bind to the sperm. There is no need to remove the excess S19 antibody before this step as it has been shown that the presence of some free antibody facilitates the crosslinking of sperm and particles and increases the number of cells that can be recovered (Hancock and Kemshead, 1993). Inserting a plate with 96 metal pins that has magnetism conferred by an adjacent fixed magnet or electromagnet into the wells would quickly pick up only the antibody coated sperm cells. The plate with sperm attached to pins would then be transferred to a new Microtitre plate containing reaction mix for the PCR analysis. By removing the source of magnetism to the pins and placing another magnet under the microtitre plate, the particle coated sperm would be released from the pins and attracted to the bottom of the wells in the plate. Buffers may be added or replaced in all of the wells using a system of positive and negative pressure pumps directing fluids through individual tubes in each well.

### EXAMPLE 7

Expression and Purification of the Recombinant Anti-Sperm Antibody (RASA).

The native S19 mAb will be grown in hybridoma tissue culture and purified for use in the sperm immunoselection device. However, production of the mAb in this manner my incur considerable expense. Production of the recombinant S19 miniantibody, RASA, in bacterial culture will provide a cost effective alternative. As described in the preliminary results, RASA has been generated in its active form in the pCANTAB/HB2151 system. In preparation for large-scale production of RASA, the expression cassette was subcloned into the pET-28b vector, an expression vector that affords high levels of recombinant protein expression. RASA will be expressed and purified in bacterial culture and the purified RASA will be tested for anti-SAGA-1 immunoreactivity.

Preparation of Expression Plasmid Construct:

To prepare the ScFv for insertion into the high expression pET-28b vector (Novagen, Madison, Wis.) the cDNA was PCR amplified with primers to introduce restriction sites for cloning. NcoI and NotI sites were added to the 5′ and 3′ ends of the cDNA fragment, respectively. The resulting ScFv cDNAs were gel purified, quantitated, and digested with NcoI and NotI to generate cohesive ends for insertion into the pET expression vector previously cut with these enzymes.

The insertion site in this plasmid incorporates an 18 bp (6 amino acid “HHHHHH”) sequence designated the His-Tag at the 3′ end of the ScFv. Antibodies to this peptide tag or nickel-charged resins can be used to identify full-length recombinant proteins. Plasmids containing the ScFv insert were transformed into BL21pLysS cells (Novagen). Transformed cells were grown on plates containing kanamycin to select for colonies containing the ScFv insert. Expressed RASA containing the His-Tag should have a molecular weight of 29 kDa.

Expression and Purification of RASA:

For large-scale purification, we will follow the methodology of Reddi, et al. (1994). 200 ml of YT media (per liter: 16 g Bacto tryptone, 10 g yeast extract, 5 g NaCl, pH 7.0) containing 50 μg/ml kanamycin will be inoculated with the transformed cells above. The culture will incubate overnight at 37° C. at 220 rpm. 10 liters of fresh YT media including 1 ml Antifoam 289 (Sigma, St. Louis, Mo.) will be inoculated with the overnight culture and incubated in a New Brunswick Microgen™ bench fermentor with an agitator rotor (New Brunswick, N.J.). The culture will be incubated at 37° C. at 400 rpm with 20 liter/min aeration. When the A_{600 nm} of the culture reaches 0.6 it will be induced by adding sterile IPTG to a final concentration of 0.4 mM. After 3 hours the culture will be harvested into liter bottles by centrifugation at 3300 rpm in a Beckman J6M centrifuge for 10 min. at 4° C. The resulting pellet will be resuspended in 200 ml of binding buffer (5 mM imidazole, 0.5 mM NaCl, 20 mM Tris-HCl pH 7.9) and sonicated for 8 minutes to disrupt the bacterial cells. The sonicate will be centrifuged at 20,000× g for 15 min. and the supernatant (soluble extract) tested for the presence of RASA.

Should RASA be insoluble in this cell line the pellet from the centrifugation step will be washed with 100 ml binding buffer and re-centrifuged. This pellet will then be dissolved in 250 ml binding buffer containing 6 M Gu-HCl, incubated on ice for 1 hour and centrifuged at 39,000× g for 20 min at 4° C. The supernatant (insoluble fraction) will be collected and filtered through a 0.45 micron filter. 125 ml of His-Bind™ resin (Novagen) will be rinsed with 375 ml distilled water and then charged with 625 ml of 50 mM NiSO₄. The charged beads will be rinsed with 375 ml binding buffer containing 6 M Guanidine-HCl (Gu-HCl) pH 7.9. The filtered cell extract will be bound to the resin via the His-Tag and washed first with 1250 ml of binding buffer containing 6 M Gu-HCl pH 7.9, followed by 375 ml wash buffer (20 mM imidazole, 0.5 M NaCl, 20 mM Tris-HCl pH 7.9 and 6 M GuHCl). The RASA will be eluted with 750 ml elution buffer (1 M imidazole, 0.5 M NaCl, 20 mM Tris-HCl pH 7.9 and 6 M Gu-HCl). The eluate will be extensively dialyzed against PBS. The dialyzed material will then be concentrated by ultrafiltration in a 150 ml Filtron stirred cell unit with a 10 kDa MWCO Omega series filter. The concentration of purified RASA will be determined using the Micro BCA Assay (Pierce, Rockford, Ill.). As the final concentration will likely amount to less than 1 gram of RASA, the appropriate number of subsequent fermentor batches will be run to accumulate 1 gram of total material.

Evaluation of RASA for Anti-SAGA-1 Activity:

Western Blot Analysis:

Each His-tagged recombinant protein preparation will be tested for binding to the SAGA-1 antigen by Western blot analysis, as above. Ejaculated human sperm will be separated by SDS-PAGE, and transferred to nitrocellulose. Blots will be incubated with recombinant antibodies followed by incubation with an enzyme-conjugated Ni-NTA (which binds the His-Tag). Native S19 mAb and Ni-NTA alone will serve as positive and negative controls, respectively. immunoreactivity with SAGA-1 on immunoblots will be identified by the development of dark blue bands using enzyme substrate TMB. Both the original S19 mAb and RASA should identify an identical set of bands ranging from 15-25 kDa.

Immunofluorescence Analysis:

To confirm that the RASA made in the BL21pLysS cells retains activity with the sperm surface we will perform immunofluorescence on viable human sperm (Homyk and Herr 1993), using an anti-His tag antibody as opposed to the anti-E Tag antibody used previously. This analysis will confirm that the recombinant miniantibody made in BL21pLysS binds the entire human sperm surface.

Agglutination Assay:

Agglutination of human spermatozoa by RASA will be investigated by generating a small batch of magnetic particles (beads or filaments) coated with RASA. The agglutination of spermatozoa will indicate that the new RASA formulation binds spermatozoa and will be appropriate for use as a sperm cell selection agent.

### EXAMPLE 8

Evaluating and Quantifying the Efficiency of Methods for Separating Sperm DNA from DNA of Contaminating Cellular Sources Using the Sperm Immunoselective Device.

To develop methods to separate sperm from either dried stains on clothing or on swabs stored at room temperature, and from swabs that have not been allowed to dry but stored at 4° C. the following experiments will be utilized. Swabs from female autopsies will be used to collect vaginal epithelial cells and fluid for analysis. Additional material will be collected by lavage with physiological saline. In addition swabs of cheek epithelial cells will be obtained from female volunteers. In the final phase of the study, post coital vaginal swabs will be collected from 10 volunteer couples.

For analysis of fresh samples, forensic evidence will be simulated using Scopette cotton swabs saturated with female epithelial cells and fluid from one of the sources above. One swab will be used as a source for the female component cells for PCR analysis. Semen will be obtained from volunteers as currently approved by the Human Investigations Committee. Other swabs will be soaked in 1 ml semen diluted with phosphate buffered saline (PBS) pH 7.2 to a range of sperm concentrations from 10² per ml to 10⁵ per ml. Sperm will be separated from the resulting mixture of sperm and female epithelial cells using the S19-paramagnetic beads to be evaluated. An aliquot of washed sperm from the same donor will provide the positive control for the PCR analysis of the immunoselected sperm. The sperm concentration for the sample recovered with the immunoselective device will be determined in order to calculate the efficiency of recovery of sperm. After isolation of the DNA the amount of sperm DNA recovered will be calculated from the absorbance at 260 nm of the solution. Final proof of the ability of the immunoselective device to separate sperm from female component cells will be demonstrated by an ethidium bromide-stained agarose gel with three lanes of PCR products: 1) from female component cells, 2) from sperm immunoselected from the mix, and 3) from the washed sperm aliquot.

The efficiency of extraction of sperm from dried swabs stored at room temperature will also be tested. Swabs will be saturated with semen that has been diluted to a range of concentrations from 10² to 10⁵ per ml with PBS from a lavage. They will be dried and then stored at room temperature for varying periods of time up to 3 months. Methods will be devised for releasing the sperm from the dried swab, including soaking in PBS with 0.05% Tween 20 for 2 hours and physically teasing apart the fibers of the swab. The S19 immunoselective particles will be used to separate the sperm from other cellular material and the efficiency of sperm recovery determined by sperm count and subsequent DNA isolation.

Recovery of sperm from dried fabric stains will also be evaluated. Fabric stains will be made by applying a known concentration of sperm diluted in lavage PBS to a piece of cotton cloth and allowing it to dry at room temperature. Fabric with dried stains will be scrubbed with a small brush in PBS and then soaked with gentle agitation overnight at 4° C. in PBS containing 0.05% Tween 20 (Giusti et al. 1986). Sperm will be separated from other cellular components with the S19-immunoselective device and the efficiency of sperm recovery determined by sperm count and subsequent DNA isolation.

Determining Sperm Concentration:

10 ul of the sperm suspension recovered above will be placed under the cover glass on each side of a Neubauer hemacytometer. The sperm will be allowed to settle onto the hemacytometer for 10 minutes before counting the number of sperm in 5 squares (0.2 mm on a side) in a diagonal line on the grid. The sperm concentration per ml will be reported as the average of the counts from both sides of the hemacytometer×10⁶ (WHO Laboratory Manual 1992, Howards 1994).

DNA Analysis with PCR Using Short Tandem Repeat Loci.:

PCR using short tandem repeat loci (STR) has proved to be a high stringency method for identifying individuals. These loci are simple tandemly repeated sequences of 1-6 base pairs (bp) in length which vary among individuals in the number of repeats displayed. Repeated sequences can be identical (simple) or complex. STRs appear in the genome every 6-10 kilo bp and are easily identifiable through PCR analysis. As PCR is a procedure that utilizes amplification, it requires very little DNA starting material. Fluorescent primers to STRs located on various chromosomes generate discreet bands that are specific to an individual. Fluorescence-based PCR technology followed by automated sequence analysis provide the tools for creating a DNA fingerprint for an individual.

PCR Testing Procedure:

The same procedure as described in Example 4 will be used.

1. A method of purifying sperm cell DNA from a sample comprising sperm cells and other cell types, said method comprising the steps of
providing a sample vessel that contains the sample;
contacting the sample with a binding substrate for a time sufficient to allow cells present in the sample to bind to the binding substrate, said binding substrate comprising an antibody against a sperm specific protein and a solid support, wherein said antibody is linked to the solid support;
washing the binding substrate with a wash solution;
separating the binding substrate from the wash solution;
lysing the sperm cells bound to the antibody; and
purifying the sperm cell DNA.

2. The method of claim 1 wherein the solid support is in particulate form and has a diameter ranging from about 120 to about 150 nm.

3. The method of claim 1 wherein the solid support is a magnetic particle.

4. The method of claim 3 wherein the step of separating the binding substrate from the wash solution comprises the steps of applying a magnetic field to the sample to immobilize the magnetic particles and removing the non-immobilized material.

5. The method of claim 4 wherein the magnetic particles are immobilized on the interior surface of the sample vessel, and the step of removing the non-immobilized material comprises aspiration of non-immobilized material.

6. The method of claim 4 wherein the step of removing the non-immobilized material comprises removing the immobilized magnetic particles from the sample vessel and releasing the magnetic particles into a new vessel.

7. The method of claim 4 wherein the antibody is MHS 10, 6C12, S-19, 3C12 or an antibody that binds to SPAN-X.

8. The method of claim 7 wherein the binding substrate comprises a plurality of antibodies, wherein each antibody binds to a different sperm specific protein.

9. The method of claim 4 wherein the antibody is a monoclonal antibody that binds to a protein selected from the group consisting of SP 10 and SPAN-X.

10. A device for isolating sperm cell DNA from a sample comprising sperm cells and other cell types, said device comprising
an electromagnet;
a robotic arm coupled to said electromagnet;
a first and second compartment, wherein the first compartment is formed for receiving said sample and the second compartment is formed for receiving a cell lysis solution; and
automated means for moving the electromagnet from the first compartment to the second compartment.

11. The device of claim 10 further comprising a magnetic pin magnetically coupled to said electromagnet.

12. The device of claim 11 further comprising a source of magnetism located in close proximity to the second compartment.

13. The device of claim 12 further comprising automated means for dispensing liquid into and withdrawing liquid from the first and second compartment.

14. The device of claim 10 further comprising a third chamber formed for receiving a wash solution and wherein said automated means move the electromagnet sequentially from the first compartment to the third compartment and then to the second compartment.

15. The device of claim 10 wherein the second compartment comprises a prepackaged cell lysis solution.

16. The device of claim 14 wherein the second compartment comprises a prepackaged cell lysis solution.

17. A device for isolating sperm cell DNA from a sample comprising sperm cells and other cell types, said device comprising
a vessel for holding a biological sample;
an electromagnet in contact with an exterior surface of said vessel;
automated means for removing fluids from the vessel; and
automated means for adding a cell lysis solution to said vessel.

18. A method for isolating a single sperm cell from a sample comprising a mixture of cells, said method comprising the steps of
contacting the sample with a binding substrate for a time sufficient to allow cells present in the sample to bind to the binding substrate, said binding substrate comprising an antibody against a sperm specific protein and a magnetic particle, wherein said antibody is linked to the magnetic particle;
microscopically observing the sample to identify separate sperm cells;
contacting a single sperm cell with a magnetized probe;
placing the probe into a reaction vessel and deactivating the magnetized probe.

19. The method of claim 18 wherein an exterior wall of the reaction vessel is placed in contact with a second source of magnetism when the magnetized probe is deactivated.

20. A composition for isolating sperm cells from a suspension of cells released from a swab collected as evidence, said composition comprising a plurality of antibodies that bind to different sperm specific proteins, wherein the sperm specific proteins are selected from the group consisting of SP-10, SAGA-1 and SPAN-X, and said antibodies are linked to a solid support.
